(19) United States
(12) Patent Application Publication
(10) Pub. No.: US 2023/0256142 A1
(43) Pub. Date: Aug. 17, 2023

(54) ABSORBABLE IRON-BASED ALLOY IMPLANTED MEDICAL DEVICE

(71) Applicant: Biotyx Medical (Shenzhen) Co., Ltd., Shenzhen (CN)

(72) Inventors: Deyuan ZHANG, Shenzhen (CN); Haiping QI, Shenzhen (CN); Wenjiao LIN, Shenzhen (CN); Gui ZHANG, Shenzhen (CN); Li QIN, Shenzhen (CN)

(73) Assignee: Biotyx Medical (Shenzhen) Co., Ltd., Shenzhen (CN)

(21) Appl. No.: 18/304,485

(22) Filed: Apr. 21, 2023

Related U.S. Application Data
(63) Continuation-in-part of application No. 18/297,898, filed on Apr. 10, 2023, now abandoned.
Application No. 18/297,898 is a continuation of application No. 15/766,842, filed on Apr. 9, 2018, now Pat. No. 11,623,028.

(30) Foreign Application Priority Data
Dec. 22, 2015 (CN) 201510979056.0

(51) Int. Cl. A61L 31/02; A61L 31/14; A61L 31/10; A61L 31/06; A61L 31/16

(52) U.S. Cl. CPC A61L 31/022; A61L 31/14; A61L 31/10; A61L 31/06; A61L 31/148; A61L 31/16; A61L 2300/41; A61L 2300/416; A61L 2300/42; A61L 2400/18

(57) ABSTRACT

An absorbable implantable medical device made of iron-based alloy, including a base made of iron-based alloy and a complex, wherein the complex includes a complexing agent. In a physiological solution, the base made of iron-based alloy can react with the complexing agent to generate a water-soluble iron complex having solubility in the physiological solution of no less than 10 mg/L. A corrosion product generated after the absorbable implantable medical device made of iron-based alloy is implanted in a human body can be quickly metabolized/absorbed by the body.

### TECHNICAL FIELD

The embodiments belong to the field of absorbable implanted medical devices, and relates to an absorbable iron-based alloy implanted medical device.

### BACKGROUND

At present, matrix materials for an absorbable implanted medical device mainly include polymers, a magnesium-based alloy and an iron-based alloy. The most frequently applied polymer is polylactic acid, which can be completely degraded and absorbed, with degradation products of carbon dioxide and water, but its mechanical property is poor. The size of the polymer-based device should be larger than the metal-based device so that the polymer-based device has the same mechanical properties as the metal-based device, which limits application of the polymer-based device. The magnesium-based alloy and the iron-based alloy have advantages of easiness in processing and molding and high mechanical strength. However, as the magnesium-based alloy is corroded too fast in a human body, it is necessary to enlarge the size of a magnesium-based alloy device to obtain the mechanical property in the early stage of implantation, and in this way, the application of the magnesium-based alloy is limited as well.

As a matrix material of the implanted medical device, the iron-based alloy has a good biological compatibility, and its corrosion speed is lower than that of the magnesium-based alloy. On the premise of obtaining the same mechanical property in the early stage of implantation, the size of the iron-based alloy is smaller than the polymer-based device and the magnesium-based alloy device. However, an insoluble corrosion product would be generated by corrosion of the iron-based alloy material in the body. The volume of the corrosion product is generally 3 to 8 times that of the iron-based alloy substrate and the corrosion product is hardly quickly absorbed/metabolized by an organ, which possibly leads to some potential biology risks. Therefore, it needs to reduce insoluble corrosion products for an iron-based alloy implanted medical device.

### SUMMARY

For shortcomings in the prior art, the embodiments provide an absorbable iron-based alloy implanted medical device. After the absorbable iron-based alloy implanted medical device is implanted into a body, all/part of corrosion products of an iron-based alloy are turned into water-soluble iron complexes which are quickly metabolized/absorbed by an organ.

An absorbable iron-based alloy implanted medical device is provided, including an iron-based alloy substrate and a complex body. The complex body includes a complexing agent. In a physiological solution, the iron-based alloy substrate may react with the complexing agent to generate a water-soluble iron complex which has a solubility (metered by iron) greater than or equal to 10 mg/L in the physiological solution.

The complex body may consist of a complexing agent, or further includes an adhesive and/or a thickener besides the complexing agent. When the complex body contains other components besides the complexing agent, the volume percent of the complexing agent is greater than or equal to 10 percent but less than 100 percent.

The complexing agent is a strong field monodentate ligand and/or a polydentate ligand.

The strong field monodentate ligand contains a coordination group which is a cyanide group, thiocyanate (S—C≡N<->), iso-thiocyanate (N═C═S<->) or nitryl (—NO₂).

The cyanide group-containing monodentate ligand is selected from the group consisting of sodium cyanide, zinc cyanide, 3-cyanopyridine, dicyandiamide and palmitonitrile; the thiocyanate-containing monodentate ligand is selected from the group consisting of sodium hydrosulfide, potassium thiocyanate and calcium thiocyanate; the iso-thiocyanate-containing monodentate ligand is selected from the group consisting of potassium isothiocyanate and isothiocyanate; and the nitryl-containing monodentate ligand is selected from the group consisting of nitrocyclopentane and 2-bromo-2-nitro-1,3-propanediol.

The polydentate ligand contains at least two coordination groups selected form the group consisting of hydroxyl on polycyclic aromatic hydrocarbon, sulfydryl (—SH), amido

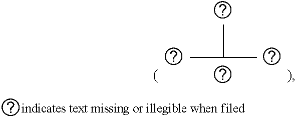

a hetero aromatic group, nitroso (O═N—), carbonyl

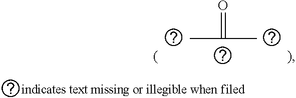

sulpho

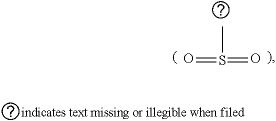

a phosphate group

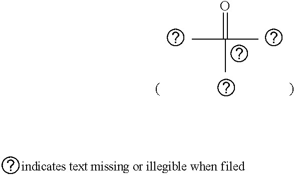

and an organic phosphorus group

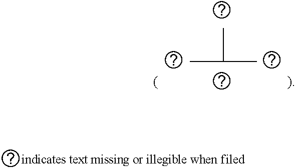

The hydroxyl on the polycyclic aromatic hydrocarbon is a phenolic hydroxyl group. The hetero aromatic group is selected from the group consisting of furyl

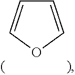

pyrrl

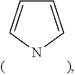

thienyl

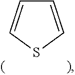

imidazolyl

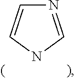

triazolyl

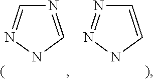

thiazolyl

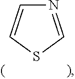

pyridyl

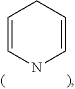

a pyridone group

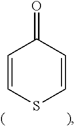

pyranyl

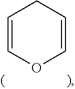

a pyrone group

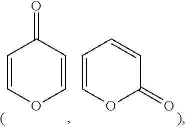

pyrimidyl

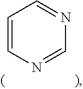

pyridazinyl

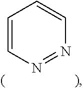

pyrazinyl

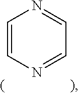

quinolyl

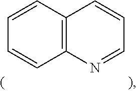

isoquinolyl

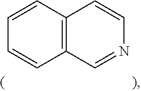

phthalazinyl

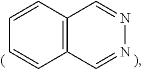

pteridyl

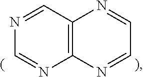

indolyl

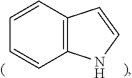

purinyl

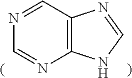

and a phenanthroline group

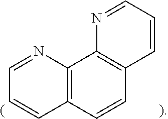 [SMILES: c1cnc2c(c1)ccc1cccnc12]

The hydroxyl-on-polycyclic aromatic hydrocarbon-containing polydentate ligand is selected from the group consisting of 8-hydroxyquinoline, 8-hydroxyquinaldine, 4,5-dioxybenzene-1,3-sodium disulfonate, 4-[3,5-bis-hydroxyphenyl- 1H-1,2,4-triazole]-benzoic acid (deferasirox), or a polymers or a copolymers containing thereof; the sulfydryl-containing polydentate ligand is selected from the group consisting of 8-mercaptoquinoline, mercaptoacetic acid and 5-methyl-2 mercapto mercaptobenzoate, or a polymers or a copolymers containing thereof; the amido-containing polydentate ligand is selected from the group consisting of ethylenediamine, triethylene tetramine, ethylenediamine tetraacetic acid, ethylene diamine tetraacetic acid tetrasodium and N′-[5-[[4-[[5-(acetyl hydroxylamine)amyl]ammonia]-1,4-dioxo butyl]hydroxylamine]amyl]-N-(5-amido amyl)-N-hydroxyl succinamide (deferoxamine), or a polymers or a copolymers containing thereof; the hetero aromatic group-containing polydentate ligand is selected from the group consisting of phenanthroline, dipyridyl, porphyrin, porphin, chlorophyll, hemoglobin, 1,2-dimethyl-3-hydroxyl-4-pyridone (deferiprone), or a polymers or a copolymers containing thereof; the nitroso-containing polydentate ligand is selected from the group consisting of 1-nitroso-2-naphthol and 1-nitroso-2-naphthol-6-sodium sulfonate, or a polymers or a copolymers containing thereof; the carbonyl-containing polydentate ligand is selected from the group consisting of polybasic carboxylic acid and salt thereof, anhydride, ester, amide, polycarboxylic acid and polyanhydride; the sulpho-containing polydentate ligand is selected from the group consisting of sulfosalicylic acid, 8-hydroxyquinoline-5-sulphonic acid; the phosphate group-containing polydentate ligand is selected from the group consisting of pyrophosphoric acid, tripolyphosphoric acid, hexadecophosphoric acid, polyphosphoric acid, sodium pyrophosphate, sodium hexametaphoshpate, ammonium polyphosphate, or a polymers or a copolymers containing thereof; the organic phosphorus group-containing polydentate ligand is selected from the group consisting of potassium diethylenetriamine penta (methylene phosphonate), sodium ethylenediamine tetra (methylene phosphonate), or a polymers or a copolymers containing thereof; and the carbonyl-containing polydentate ligand is further selected from the group consisting of oxalic acid, tartaric acid, malic acid, succinic acid, oxaloacetic acid, fumaric acid, maleic acid, citric acid, nitrilotriacetic acid, diethylene triamine penta(carboxylic acid), alginic acid, maleic anhydride, glycine, alanine, valine, leucine, isoleucine, methionine, proline, tryptophan, serine, tyrosine, cysteine, phenylalanine, asparagine, glutamine, threonine, arginine, histidine, selenocysteine, pyrrolysine, glutamic acid, aspartic acid, ornithine, lysine, potassium citrate, calcium citrate, monoglyceride citrate, acetylsalicylic acid, sulpho salicylamide, or a polymers or a copolymers containing thereof, such as polyglycine, polyalanine, polyvaline, polyleucine, polyisoleucine, polymethionine, polyproline, polytryptophan, polyserine, polytyrosine, polycysteine, polyphenylalanine, polyasparagine, polyglutamine, polythreonine, polyarginine, polyhistidine, polyselenocysteine and, pyrrole lysine, polyaspartic acid, polyglutamic acid, polyornithine, poly lysine, polylysine-polyglutamic acid, polylysine-polyglycine, polylysine-polyleucine, polylysine-polyalanine, polylysine-polyglutamic acid, polyethylene glycol-polyleucine, polyethylene glycol-polyaspartic acid, polyaspartic acid-polylactic acid, polyglycine-polylactic acid, polyglycine and so on.

The adhesive is selected from at least one of polyethylene glycol, polyvinyl alcohol, starch, cyclodextrin or water-soluble inorganic salt; and the thickener is selected from at least one of gelatin, polyvinylpyrrolidone (PVP) or sodium carboxymethylcellulose (CMC).

The complex body is disposed on the surface of or inside the iron-based alloy substrate.

The implanted medical device further includes a degradable polymer layer in which an active drug is mixed, or no active drug is mixed.

The iron-based alloy substrate may be pure iron or an iron-based alloy with a carbon content less than or equal to 2.11 wt. %.

Compared with the prior art, the absorbable iron-based alloy implanted medical device includes the complex body. After the device is implanted into the body, the iron-based alloy substrate is corroded to generate Fe<2+> or Fe<3+> under a physiological environment, and complex reaction occurs between Fe<2+> or Fe<3+> and the complexing agent, thereby generating the water-soluble iron complex, and the quantity of insoluble solid corrosion products of the iron-based alloy is reduced.

### DETAILED DESCRIPTION OF THE EMBODIMENTS

In order to facilitate understanding, embodiments are provided. However, the embodiments may be implemented by many different forms, but are not limited by the embodiment described herein. On the contrary, the object of providing these embodiments is to provide more thorough and comprehensive details and examples.

Unless otherwise defined, all technical and scientific terms used herein have the same meanings of general understandings of persons skilled in the art of the embodiments. Terms used in the description herein are only intended to describe the specific embodiments, but not to limit them.

In the embodiments, an iron-based alloy substrate and a complex body react with each other in a physiological solution to generate a water-soluble iron complex to avoid or reduce generation of insoluble solid corrosion products.

After an iron-based alloy medical device is implanted into a human body, the iron-based alloy substrate is gradually corroded in the physiological solution to generate a primary corrosion product Fe<2+> or Fe<3+> which then quickly reacts with OH<-> to generate the insoluble corrosion products such as Fe(OH)2 and Fe(OH)3 and to further generate insoluble substances such as FeOOH, Fe2O3 and Fe3O4. Reaction equations are as shown in formulas from (1) to (3). With loose structures, these corrosion products may be expanded by 3 to 8 times in volume than the iron-based alloy substrate, and are really hard to dissolve in the physiological solution, so that it would take a very long time for a tissue to metabolize/absorb them, and they would be retained in the tissue for a long time, which may possibly lead to some potential biology risks.

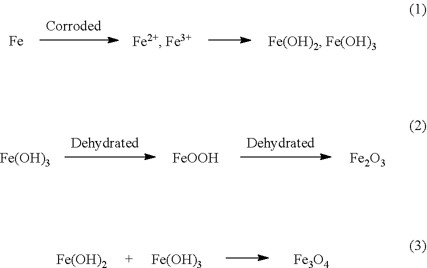

The complexing agent is also called a ligand, which structurally contains a coordination group for lone pair electrons or n electrons. In order to reduce generation of the iron insoluble corrosion products, the complexing agent (which is also called the ligand, and abbreviated as L) is disposed in the iron-based alloy implanted medical device. Under a physiological environment, the complexing agent may provide the lone pair electrons or n electrons for complex reaction with Fe<2+> and/or Fe<3+> to generate a water-soluble iron complex. The water-soluble iron complex may be metabolized/absorbed by an organ more quickly than the insoluble solid corrosion products, and its stability is higher than that of Fe<OH>2 and/or Fe(OH)3 and would not be turned into insoluble Fe<OH>2 and/or Fe(OH)3 in the physiological solution. Its reaction equation is as shown in formula (4).

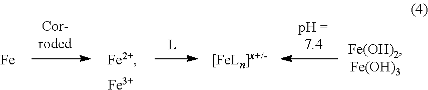

It is defined that the iron complex, of which the solubility in the physiological environment is not lower than 10 mg/L, is called the water-soluble iron complex. In case of the solubility greater than or equal to 100 mg/L in the physiological environment, the iron complex may be quickly diffused and metabolized; in case of the solubility greater than or equal to 10 mg/L but less than 100 mg/L in the physiological environment, although the solubility of such iron complex is lower, complex sedimentation may occur due to saturation of the iron complex concentration in the solution, complex sediments may be continuously and gradually dissolved with gradual diffusion and absorption/metabolism of the dissolved iron complex in the physiological solution, and are finally completely dissolved and diffused and then absorbed/metabolized by the tissue.

The complexing agent is a strong field monodentate ligand and/or a polydentate ligand.

The monodentate ligand easily provides the lone pair electrons which form an inner-orbital complex together with Fe<2+>/Fe<3+>. The inner-orbital complex has a mechanism which is more stable than an outer-orbital complex and may not be turned into an insoluble substance Fe(OH)2 and/or Fe(OH)3 under a physiological condition.

The monodentate ligand contains a coordination group which is a cyanide group, thiocyanate iso-thiocyanate (N═C═S<->) or nitryl (—NO2).

The cyanide group-containing monodentate ligand is selected from the group consisting of sodium cyanide, zinc cyanide, 3-cyanopyridine, dicyandiamide and palmitonitrile; the thiocyanate-containing monodentate ligand is selected from the group consisting of sodium hydrosulfide, potassium thiocyanate and calcium thiocyanate; the iso-thiocyanate-containing monodentate ligand is selected from the group consisting of potassium isothiocyanate and isothiocyanate; and the nitryl-containing monodentate ligand is selected from the group consisting of nitrocyclopentane and 2-bromo-2-nitro-1,3-propanediol.

The polydentate ligand contains at least two coordination groups which may form a more stable chelate (an annular structure) together with ions Fe<2+>/Fe<3+>.

The polydentate coordination group is selected from the group consisting of hydroxyl on polycyclic aromatic hydrocarbon, sulfydryl (—SH), amido

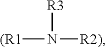

a hetero aromatic group, nitroso (O═N—), carbonyl

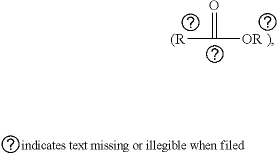

sulpho

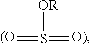

a phosphate group

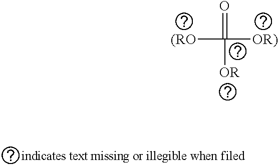

and an organic phosphorus group

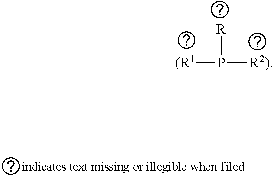

The polydentate ligand may either contain at least two identical coordination groups, or contain different coordination groups.

The hydroxyl on the polycyclic aromatic hydrocarbon is a phenolic hydroxyl group. The hetero aromatic group is selected from furyl

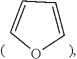

pyrryl

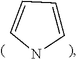

thienyl

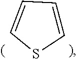

imidazolyl

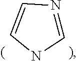

triazolyl

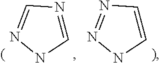

thiazolyl

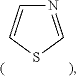

pyridyl

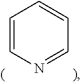

a pyridone group

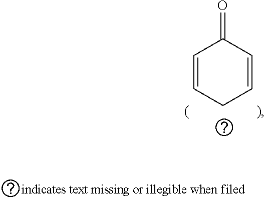

pyranyl

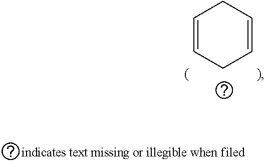

a pyrone group

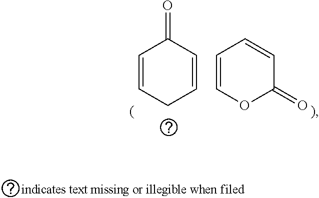

pyrimidyl

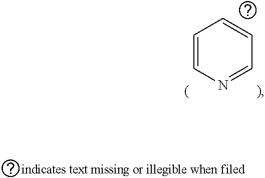

pyridazinyl

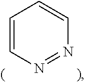

pyrazinyl

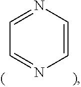

quinolyl

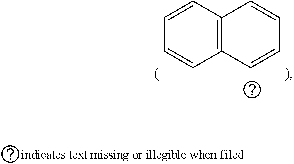

isoquinolyl

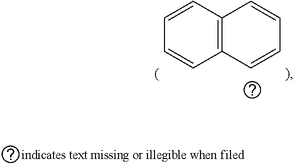

phthalazinyl

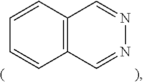

pteridyl

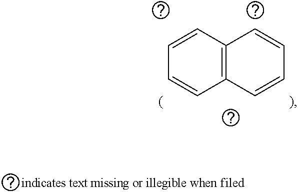

indolyl

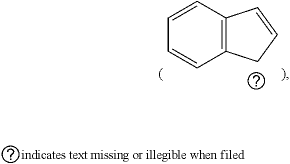

purinyl

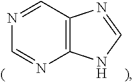

and a phenanthroline group

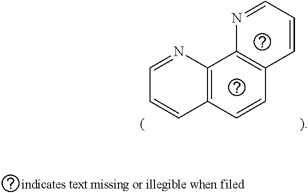 [SMILES: c1cnc2c(c1)ccc1cccnc12]

The hydroxyl-on-polycyclic aromatic hydrocarbon-containing polydentate ligand is selected from the group consisting of 8-hydroxyquinoline, 8-hydroxyquinaldine,4,5-dioxybenzene-1,3-sodium disulfonate, 4-[3,5-bis-hydroxyphenyl-1H-1,2,4-triazole]-benzoic acid (deferasirox), or a polymers or a copolymers containing thereof; the sulfydryl-containing polydentate ligand is selected from the group consisting of 8-mercaptoquinoline, mercaptoacetic acid and 5-methyl-2 mercapto mercaptobenzoate, or a polymers or a copolymers containing thereof; the amido-containing polydentate ligand is selected from the group consisting of ethylenediamine, triethylene tetramine, ethylenediamine tetraacetic acid, ethylene diamine tetraacetic acid tetrasodium and N′-[5-[[4-[[5-(acetyl hydroxylamine)amyl]ammonia]-1,4-dioxo butyl]hydroxylamine]amyl]-N-(5-amido amyl)-N-hydroxyl succinamide (deferoxamine), or a polymers or a copolymers containing thereof; the hetero aromatic group-containing polydentate ligand is selected from the group consisting of phenanthroline, dipyridyl, porphyrin, porphin, chlorophyll, hemoglobin, 1,2-dimethyl-3-hydroxyl-4-pyridone (deferiprone), or a polymers or a copolymers containing thereof; the nitroso-containing polydentate ligand is selected from the group consisting of 1-nitroso-2-naphthol and 1-nitroso-2-naphthol-6-sodium sulfonate, or a polymers or a copolymers containing thereof; the carbonyl-containing polydentate ligand is selected from the group consisting of polybasic carboxylic acid and salt thereof, anhydride, ester, amide, polycarboxylic acid and polyanhydride; the sulpho-containing polydentate ligand is selected from the group consisting of sulfosalicylic acid, 8-hydroxyquinoline-5-sulphonic acid; the phosphate group-containing polydentate ligand is selected from the group consisting of pyrophosphoric acid, tripolyphosphoric acid, hexadecophosphoric acid, polyphosphoric acid, sodium pyrophosphate, sodium hexametaphoshpate, ammonium polyphosphate, or a polymers or a copolymers containing thereof; the organic phosphorus group-containing polydentate ligand is selected from the group consisting of potassium diethylenetriamine penta (methylene phosphonate), sodium ethylenediamine tetra (methylene phosphonate), or a polymers or a copolymers containing thereof; and the carbonyl-containing polydentate ligand is further selected from the group consisting of oxalic acid, tartaric acid, malic acid, succinic acid, oxaloacetic acid, fumaric acid, maleic acid, citric acid, nitrilotriacetic acid, diethylene triamine penta(carboxylic acid), alginic acid, maleic anhydride, glycine, alanine, valine, leucine, isoleucine, methionine, proline, tryptophan, serine, tyrosine, cysteine, phenylalanine, asparagine, glut amine, threonine, arginine, histidine, selenocysteine, pyrrolysine, glutamic acid, aspartic acid, ornithine, lysine, potassium citrate, calcium citrate, monoglyceride citrate, acetylsalicylic acid, sulpho salicylamide, or a polymers or a copolymers containing thereof, such as polyglycine, polyalanine, polyvaline, polyleucine, polyisoleucine, polymethionine, polyproline, polytryptophan, polyserine, polytyrosine, polycysteine, polyphenylalanine, polyasparagine, polyglutamine, polythreonine, polyarginine, polyhistidine, polyselenocysteine and, pyrrole lysine, polyaspartic acid, polyglutamic acid, poly ornithine, polylysine, polylysine-polyglutamic acid, polylysine-polyglycine, polylysine-polyleucine, polylysine-polyalanine, polylysine-polyglutamic acid, polyethylene glycol-polyleucine, polyethylene glycol-polyaspartic acid, polyaspartic acid-polylactic acid, polyglycine-polylactic acid, polyglycine and so on.

The complex body may further include an adhesive and/or a thickener. When the complex body is a complexing agent and adhesive and/or thickener mixture, the volume percent of the complexing agent in the mixture is greater than or equal to 10 percent but less than 100 percent. The adhesive is selected from at least one of polyethylene glycol, polyvinyl alcohol, starch, cyclodextrin or water-soluble inorganic salt; and the thickener is selected from at least one of gelatin, polyvinylpyrrolidone (PVP) or sodium carboxymethylcellulose (CMC). The adhesive makes firmer combination between the complexing agent and the substrate, and the thickener may achieve a slow release effect on the complexing agent.

The amount (weight or volume) of the complex body may be flexibly selected according to the type and the specification of the device and the solubility of the formed iron complex, thereby adjusting the amount of the iron complex generated by reaction with Fe<2+> or Fe<3+> formed by corrosion of the iron-based alloy substrate, and fulfilling the aim of adjusting a metabolism/absorption cycle of a corrosion product.

The complex body may be disposed on the surface of or inside the iron-based alloy substrate in ways of spray coating, dip coating, brush coating, electrostatic spinning, 3D printing, embedding, filling and the like.

The implanted medical device further includes a degradable polymer layer. The degradable polymer layer is either disposed on the surface of the iron-based alloy substrate or embedded inside the iron-based alloy substrate. The degradable polymer is selected from degradable polyester and/or degradable polyanhydride. The degradable polyester is any one of polylactic acid, polyglycolic acid, poly(lactic acid-glycolic acid), polycaprolactone, polyhydroxyalkanoate, polyacrylate, poly(ethylene succinate), poly(β-hydroxybutyrate) and polyethylene glycol adipate, or is a physical blend of at least two of the polylactic acid, the polyglycolic acid, the poly(ethylene succinate), the poly(β-hydroxybutyrate), the polycaprolactone, the polyethylene glycol adipate, a polylactic acid-glycollic acid copolymer and a polyhydroxybutyrate-pentanoate copolymer, or is any one of copolymers formed by copolymerizing at least two of monomers forming the polylactic acid, the polyglycolic acid, the poly(ethylene succinate), the poly(β-hydroxybutyrate), the polycaprolactone, the polyethylene glycol adipate, the polylactic acid-glycollic acid copolymer and the polyhydroxybutyrate-pentanoate copolymer. The degradable polyanhydride is selected from at least one of poly1,3-bis(p-carboxyphenoxy)propane-sebacic acid, poly(erucic acid dimer-sebacic acid) or poly(fumaric acid-sebacic acid), or is a physical blend of at least two of the poly1,3-bis(p-carboxyphenoxy)propane-sebacic acid, the poly(erucic acid dimer-sebacic acid) or the poly(fumaric acid-sebacic acid), or any one of copolymers formed by copolymerizing at least two of monomers forming the poly1,3-bis(p-carboxyphenoxy)propane-sebacic acid, the poly(erucic acid dimer-sebacic acid) or the poly(fumaric acid-sebacic acid); or the degradable polymer is a copolymer formed by copolymerizing at least two of monomers forming the above degradable polyester and the above degradable polyanhydride.

The degradable polymer layer further includes an active drug, thus a curative drug may be released in a degradation process. The active drug may be a drug for inhibiting vascular proliferation, such as taxol, sirolimus and its derivative, or an antiplatelet drug such as cilostazol, or an antithrombotic drug such as heparin, or an anti-inflammatory reaction drug such as dexamethasone, or an antisensitization drug such as calcium gluconate, chlortrimeton and cortisone. The active drug also may be a mixture of at least two of the above drugs.

The iron-based alloy substrate may be pure iron or an iron-based alloy with a carbon content less than or equal to 2.11 wt. %, such as a product obtained by nitriding and/or carburizing the pure iron.

The absorbable iron-based alloy implanted medical device may be a blood vessel stent, an orthopedic implant, a gynecological implant, an andrological implant, a respiratory implant or an orthopedic implant.

By taking an iron-based alloy bone nail and an iron-based alloy coronary artery stent as examples, a further detailed description is made to the embodiments as follows, but not intended to limit the scope of the embodiments.

In a physiological solution, which may be a saturated solution or a non-saturated solution, with a pH value of 7.4 at 37° C., after an iron complex is corroded in vitro for one week, its concentration in the physiological environment is more than or equal to 10 mg/L, which indicates that the solubility of the iron complex is more than or equal to 10 mg/L. The concentration of the iron complex in the physiological environment is measured in a way as follows: under a condition of 37° C., soaking a complex body-containing medical device in a phosphate buffer solution (PBS), which is 5 times the volume of the device, for an in-vitro corrosion experiment; after the medical device is corroded in the PBS for one week, filtering the soaking solution with a water-based film having an aperture of 0.22 μm; and then testing the mass concentration of an iron element dissolved in filtrate with an atomic absorption spectrometer (AAS), thereby obtaining the concentration of the iron complex in the physiological environment.

It should be noted that each embodiment as follows, a normal fluctuation of the own performance of a product within a designed allowable range, a difference of an individual corrosion speed of the device and a system error unavoidably introduced by test ways may lead to fluctuations of detected concentrations of the iron complex within a certain range in an actual test.

### Embodiment 1

A stent is manufactured in a way of 3D printing. A material for a stent substrate is pure iron, and a groove is formed in the surface of the stent and is filled with an ethylene diamine tetraacetic acid tetrasodium and polyvinylpyrrolidone mixed coating, wherein a mass ratio of ethylene diamine tetraacetic acid tetrasodium to polyvinylpyrrolidone to iron is 0.1 to 0.1 to 1; and the outermost layer of the stent is coated with a poly-dl-lactic acid coating which has a molecular weight of 200,000 and a thickness of 4 μm. The stent is soaked in the PBS, which is 5 times the volume of the stent, at 37 s° C. . After the stent is corroded in vitro for one week, a formed iron complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the iron complex in the physiological environment is 186 mg/L.

### Embodiment 2

Micro pores in the surface of a hollow pure iron bone nail are filled with sodium pyrophosphate to manufacture an absorbable iron-based bone nail. A mass ratio of the sodium pyrophosphate to iron is 0.5 to 1. The bone nail is soaked in the PBS, which is 5 times the volume of the bone nail, at 37° C. . After the bone nail is corroded in vitro for one week, a formed ferric pyrophosphate complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the ferric pyrophosphate complex in the physiological environment is 24 mg/L.

### Embodiment 3

The surface of a pure iron stent is coated with a calcium citrate and polyethylene glycol mixed coating, which has a thickness of 5 μm, in a way of spraying coating, and a mass ratio of calcium citrate to polyethylene glycol is 2 to 1. Then a poly-dl-lactic acid-ethyl acetate (-sirolimus) solution having a molecular weight of 200,000 completely covers the surface of the stent in the way of spray coating, and after the surface is dried, an absorbable iron-based alloy stent with a poly-dl-lactic acid (-sirolimus) coating having a thickness of 5 μm is manufactured, wherein a mass ratio of poly-dl-lactic acid to sirolimus is 4 to 1. The stent is soaked in the PBS, which is 5 times the volume of the stent, at 37° C. . After the stent is corroded in vitro for one week, a formed iron gluconate complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the ferric citrate complex in the physiological environment is 113 mg/L.

### Embodiment 4

Micro pores in the surface of a hollow pure iron bone nail are filled with a palmitonitrile and polyvinylpyrrolidone mixture to manufacture an absorbable iron-based bone nail. A mass ratio of palmitonitrile to polyvinylpyrrolidone to iron is 0.25 to 0.25 to 1. The bone nail is soaked in the PBS, which is 5 times the volume of the bone nail, at 37° C. . After the bone nail is corroded in vitro for one week, a formed palmitonitrile iron complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the palmitonitrile iron complex in the physiological environment is 47 mg/L.

### Embodiment 5

The surface of a pure iron stent is coated with an acetylsalicylic acid-chloroform solution in a way of spray coating, and after the surface is dried, an acetylsalicylic acid coating having a thickness of 5 μm is manufactured, which completely covers the surface of the stent. Then a poly-dl-lactic acid-ethyl acetate (-sirolimus) solution having a molecular weight of 200,000 completely covers the surface of the acetylsalicylic acid coating in the way of spray coating; after the surface is dried, an absorbable iron-based alloy stent with a poly-dl-lactic acid (-sirolimus) coating having a thickness of 6 μm is manufactured, wherein a mass ratio of poly-dl-lactic acid to sirolimus is 4 to 1. The stent is soaked in the PBS, which is 5 times the volume of the stent, at 37° C. . After the stent is corroded in vitro for one week, a formed iron acetylsalicylate complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the iron acetylsalicylate complex in the physiological environment is 762 mg/L.

### Embodiment 6

Micro pores in the surface of a hollow pure iron bone nail are filled with a sodium ethylenediamine tetramethylene phosphonate and polyvinylpyrrolidone mixture to manufacture an absorbable iron-based bone nail. A mass ratio of sodium ethylenediamine tetramethylene phosphonate to polyvinylpyrrolidone to iron is 0.4 to 0.1 to 1. The bone nail is soaked in the PBS, which is 5 times the volume of the bone nail, at 37° C. . After the bone nail is corroded in vitro for one week, a formed iron ethylenediamine tetramethylene phosphonate complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the iron ethylenediamine tetramethylene phosphonate complex in the physiological environment is 206 mg/L.

### Embodiment 7

A hollow pure iron duct is filled with a phenanthroline and sodium carboxymethylcellulose mixture, a mass ratio of phenanthroline to sodium carboxymethylcellulose to iron is 0.2 to 0.1 to 1, and the iron duct is woven into a stent. Micro pores are formed in the surface of the stent, and the surface is coated with a poly-dl-lactic acid-ethyl acetate solution, which completely covers the surface of an acetylsalicylic acid coating and has a molecular weight of 200,000, in a way of spray coating; after the surface is dried, an absorbable iron-based alloy stent with a poly-dl-lactic acid coating having a thickness of 4 μm is manufactured. The stent is soaked in the PBS, which is 5 times the volume of the stent, at 37° C. After the stent is corroded in vitro for one week, a formed phenanthroline iron complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the phenanthroline iron complex in the physiological environment is 119 mg/L.

### Embodiment 8

Micro pores in the surface of a hollow pure iron bone nail are filled with a 4,5-dioxybenzene-1,3-sodium disulfonate and sodium carboxymethylcellulose mixture to manufacture an absorbable iron-based bone nail. A mass ratio of 4,5-dioxybenzene-1,3-sodium disulfonate to sodium carboxymethylcellulose to iron is 0.4 to 0.1 to 1. The bone nail is soaked in the PBS, which is 5 times the volume of the bone nail, at 37 ° C. . After the bone nail is corroded in vitro for one week, a formed 4,5-dioxybenzene-1,3-iron disulfonate complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the 4,5-dioxybenzene-1,3-iron disulfonate complex in the physiological environment is 276 mg/L.

### Embodiment 9

The surface of a pure iron stent is coated with a layer of 5-methyl-2-methyl mercaptobenzoate and sodium carboxymethylcellulose mixed coating in a way of spray coating, and a mass ratio of 5-methyl-2-methyl mercaptobenzoate to sodium carboxymethylcellulose to iron is 0.2 to 0.1 to 1; then the outermost layer of the stent is coated with a poly-dl-lactic acid-ethyl acetate solution which has a molecular weight of 200,000 in the way of spray coating; after the outermost layer is dried, an absorbable iron-based stent with a poly-dl-lactic acid coating having a thickness of 4 μm is manufactured. The stent is soaked in the PBS, which is 5 times the volume of the stent, at 37° C. . After the stent is corroded in vitro for one week, a formed iron complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the iron complex in the physiological environment is 89 mg/L.

### Embodiment 10

A groove is formed in the surface of a pure iron bone nail, and then is filled with a 1-nitroso-2-naphthol-6-sodium sulfonate and starch mixed coating, and the mass ratio of 1-nitroso-2-naphthol-6-sodium sulfonate to starch to iron is 0.1 to 00.1 to 1. The bone nail is soaked in the PBS, which is 5 times the volume of the bone nail, at 37 ° C. . After the bone nail is corroded in vitro for one week, a formed iron complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the 1-nitroso-2-naphthol-6-iron sulfonate complex in the physiological environment is 120 mg/L.

### Embodiment 11

Micro pores in the surface of a hollow-structured pure iron bone nail with the mass of 0.3 g are filled with a sulfosalicylic acid and polyvinylpyrrolidone mixture to manufacture an absorbable iron-based bone nail. A mass ratio of sulfosalicylic acid to polyvinylpyrrolidone to iron is 0.4 to 0.1 to 1. The bone nail is soaked in 100 mL of the PBS at 37° C. . After the bone nail is corroded in vitro for one week, a formed sulfosalicylic iron complex is fully dissolved in the solution, then the soaking solution is filtered with the water-based filtering film having the aperture of 0.22 μm, and the concentration of dissolved iron in filtrate is tested with the atomic absorption spectrometer, thereby the concentration of the sulfosalicylic iron complex in the physiological environment is 174 mg/L.

### Contrast 1

A pure iron bone nail with the mass of 1.2 g is soaked in 100 mL of the PBS at 37° C. . After the bone nail is corroded in vitro for one week, the soaking solution is filtered with the water-based filtering film having the aperture of 0.22 μm, and the concentration of dissolved iron in filtrate is tested as 0 mg/L with the atomic absorption spectrometer.

By comparison between the embodiments from 1 to 11 and Contrast 1, the absorbable implanted medical devices having the complex bodies of all the embodiments all generate the water-soluble iron complexes which has the solubility greater than 10 mg/L in the physiological environment, but the device of Contrast 1 generates an insoluble corrosion product which is hard to metabolize in the physiological environment, wherein the solubility of the iron complex generated in Embodiment 2 is relatively low, thus leading to sedimentation of part of the iron complex. However, under the physiological environment, with gradual diffusion of the dissolved iron complex, the iron complex sedimentation portion may be gradually dissolved, and are finally completely dissolved and diffused and then absorbed/metabolized. Therefore, the embodiments from 1 to 11 reduce generation of the insoluble solid corrosion products, and corrosion products of the iron-based alloy substrate are easy to metabolize/absorb.

The above embodiments only express several implementation modes of the embodiments, and their descriptions are relatively specific and detailed, but are not intended to limit the scope of the embodiments. It should be noted that a person skilled in the art can make various deformations and improvements without departing from the scope of the embodiments, and these deformations and improvements shall all fall within the scope of the embodiments.

1. An absorbable iron-based alloy implanted medical device, comprising: an iron-based alloy substrate, further comprising a complex body, wherein the complex body comprises a complexing agent; in a physiological solution, the iron-based alloy substrate reacts with the complexing agent to generate a water-soluble iron complex which has a solubility greater than or equal to 10 mg/L in the physiological solution.

2. The absorbable iron-based alloy implanted medical device according to claim 1, wherein the complex body further comprises an adhesive and/or a thickener, and the volume percent of the complexing agent is greater than or equal to 10 percent but less than 100 percent.

3. The absorbable iron-based alloy implanted medical device according to claim 1, wherein the complexing agent is a strong field monodentate ligand and/or a polydentate ligand.

4. The absorbable iron-based alloy implanted medical device according to claim 3, wherein the strong field monodentate ligand contains a coordination group which is a cyanide group, thiocyanate, iso-thiocyanate or nitryl.

5. The absorbable iron-based alloy implanted medical device according to claim 4, wherein the cyanide group-containing monodentate ligand is selected from the group consisting of: sodium cyanide, zinc cyanide, 3-cyanopyridine, dicyandiamide and palmitonitrile; the thiocyanate-containing monodentate ligand is selected from the group consisting of sodium hydrosulfide, potassium thiocyanate and calcium thiocyanate; the iso-thiocyanate-containing monodentate ligand is selected from the group consisting of potassium isothiocyanate and isothiocyanate; and the nitryl-containing monodentate ligand is selected from the group consisting of nitrocyclopentane and 2-bromo-2-nitro-1,3-propanediol.

6. The absorbable iron-based alloy implanted medical device according to claim 3, wherein the polydentate ligand contains at least two coordination groups selected from the group consisting of hydroxyl on polycyclic aromatic hydrocarbon, sulfydryl, amido, a hetero aromatic group, nitroso, carbonyl, sulpho, a phosphate group and an organic phosphorus group.

7. The absorbable iron-based alloy implanted medical device according to claim 6, wherein the hydroxyl on the polycyclic aromatic hydrocarbon is a phenolic hydroxyl group; the hetero aromatic group is selected from the group consisting of: furyl, pyrryl, imidazolyl, triazolyl, thienyl, thiazolyl, pyridyl, a pyridone group, pyranyl, a pyrone group, pyrimidyl, pyridazinyl, pyrazinyl, quinolyl, isoquinolyl, phthalazinyl, pteridyl, indolyl, purinyl and a phenanthroline group; the carbonyl-containing polydentate ligand is selected from the group consisting of polybasic carboxylic acid and salt thereof, anhydride, ester, amide, polycarboxylic acid and polyanhydride.

8. The absorbable iron-based alloy implanted medical device according to claim 6, wherein the hydroxyl-on-polycyclic aromatic hydrocarbon-containing polydentate ligand is selected from the group consisting of: 8-hydroxyquinoline, 8-hydroxyquinaldine and 4,5-dioxybenzene-1,3-sodium disulfonate, 4-[3,5-bis-hydroxyphenyl-1H-1,2,4-triazole]-benzoic acid, or a polymers or a copolymers containing thereof; the sulfydryl-containing polydentate ligand is selected from the group consisting of 8-mercaptoquinoline, mercaptoacetic acid, 5-methyl-2-mercapto mercaptobenzoate, or a polymers or a copolymers containing thereof; the amido-containing polydentate ligand is selected from the group consisting of ethidene diamine, triethylene tetramine, ethylenediamine tetraacetic acid, ethylene diamine tetraacetic acid tetrasodium, N′-[5-[[4-[[5-(acetyl hydroxylamine)amyl]ammonia]-1,4-dioxo butyl]hydroxylamine]amyl]-N-(5-amido amyl)-N-hydroxyl succinamide, or a polymers or a copolymers containing thereof; the hetero aromatic group-containing polydentate ligand is selected from the group consisting of phenanthroline, dipyridyl, porphyrin, porphin, chlorophyll, hemoglobin, 1,2-dimethyl-3-hydroxyl-4-pyridone, or a polymers or a copolymers containing thereof; the nitroso-containing polydentate ligand is selected from the group consisting of 1-nitroso-2-naphthol, 1-nitroso-2-naphthol-6-sodium sulfonate, or a polymers or a copolymers containing thereof; the sulpho-containing polydentate ligand is selected from the group consisting of sulfosalicylic acid, 8-hydroxyquinoline-5-sulphonic acid, or a polymers or a copolymers containing thereof; the phosphate group-containing polydentate ligand is selected from the group consisting of pyrophosphoric acid, tripolyphosphoric acid, hexadecophosphoric acid, polyphosphoric acid, sodium pyrophosphate, sodium hexametaphoshpate, ammonium polyphosphate, or a polymers or a copolymers containing thereof; the organic phosphorus group-containing polydentate ligand is selected from the group consisting of potassium diethylenetriamine penta(methylene phosphonate), sodium ethylenediamine tetra(methylene phosphonate), or a polymers or a copolymers containing thereof; and the carbonyl-containing polydentate ligand is further selected from the group consisting of oxalic acid, tartaric acid, malic acid, succinic acid, oxaloacetic acid, fumaric acid, maleic acid, citric acid, nitrilotriacetic acid, diethylene triamine penta(carboxylic acid), alginic acid, Maleic anhydride, Glycine, Alanine, Valine, Leucine, Isoleucine, Methionine, Proline, Tryptophan, Serine, Tyrosine, Cysteine, Phenylalanine, Asparagine, Glutamine, Threonine, Arginine, Histi dine, Selenocysteine, Pyrrolysine, glutamic acid, aspartic acid, ornithine, lysine, potassium citrate, calcium citrate, monoglyceride citrate, acetylsalicylic acid, sulpho salicylamide, or a polymers or a copolymers containing thereof.

9. The absorbable iron-based alloy implanted medical device according to claim 1, wherein the complex body is disposed on the surface of or inside the iron-based alloy substrate.

10. The absorbable iron-based alloy implanted medical device according to claim 1, further comprising a degradable polymer layer which is selected from degradable polyester and/or degradable polyanhydride; the degradable polyester is any one of polylactic acid, polyglycolic acid, poly(lactic acid-glycolic acid), polycaprolactone, polyhydroxyalkanoate, polyacrylate, poly(ethylene succinate), poly(β-hydroxybutyrate) and polyethylene glycol adipate, or is a physical blend of at least two of the polylactic acid, the polyglycolic acid, the poly(ethylene succinate), the poly(β-hydroxybutyrate), the polycaprolactone, the polyethylene glycol adipate, a polylactic acid-glycollic acid copolymer and a polyhydroxybutyrate-pentanoate copolymer, or is any one of copolymers formed by copolymerizing at least two of monomers forming the polylactic acid, the polyglycolic acid, the poly(ethylene succinate), the poly(β-hydroxybutyrate), the polycaprolactone, the polyethylene glycol adipate, the polylactic acid-glycollic acid copolymer and the polyhydroxybutyrate-pentanoate copolymer; the degradable polyanhydride is selected from at least one of poly1,3-bis(p-carboxyphenoxy)propane-sebacic acid, poly(erucic acid dimer-sebacic acid) or poly(fumaric acid-sebacic acid), or is a physical blend of at least two of the poly1,3-bis(p-carboxyphenoxy)propane-sebacic acid, the poly(erucic acid dimer-sebacic acid) or the poly(fumaric acid-sebacic acid), or any one of copolymers formed by copolymerizing at least two of monomers forming the poly1,3-bis(p-carboxyphenoxy)propane-sebacic acid, the poly(erucic acid dimer-sebacic acid) or the poly(fumaric acid-sebacic acid); or the degradable polymer is a copolymer formed by copolymerizing at least two of monomers forming the degradable polyester and the degradable polyanhydride.

11. The absorbable iron-based alloy implanted medical device according to claim 8, wherein the degradable polymer layer further comprises an active drug which is selected from the group consisting of: a drug for inhibiting vascular proliferation, an antiplatelet drug, an antithrombotic drug, an anti-inflammatory reaction drug and an antisensitization drug; the drug for inhibiting vascular proliferation is selected from at least one of taxol, sirolimus and a derivative thereof; the antiplatelet drug is cilostazol; the antithrombotic drug is heparin; the anti-inflammation reaction drug is dexamethasone; and the antisensitization drug is selected from at least one of calcium gluconate, chlortrimeton and cortisone.

12. The absorbable iron-based alloy implanted medical device according to claim 2, wherein the adhesive is selected from at least one of polyethylene glycol, polyvinyl alcohol, starch, cyclodextrin or water-soluble inorganic salt; and the thickener is selected from at least one of gelatin, polyvinylpyrrolidone (PVP) or sodium carboxymethylcellulose (CMC).

13. The absorbable iron-based alloy implanted medical device according to claim 1, wherein the iron-based alloy substrate is pure iron or an iron-based alloy with a carbon content less than or equal to 2.11 wt. %.
